(19) United States
(12) Patent Application Publication
(10) Pub. No.: US 2016/0317345 A1
(43) Pub. Date: Nov. 3, 2016

(54) A SYSTEM FOR PREVENTING SNORING AND SLEEP APNEA

(71) Applicant: Firas Marie, Magdal Shams (IL)

(72) Inventor: Firas Marie, Magdal Shams (IL)

(21) Appl. No.: 15/108,579

(22) PCT Filed: Sep. 21, 2015
(86) PCT No.: PCT/IL2015/050958

§ 371 Date: Jun. 28, 2016

(30) Foreign Application Priority Data
Oct. 21, 2014 (IL) 235257

(51) Int. Cl. A61F 5/56; A61F 2/02; A61B 17/24

(52) U.S. Cl. CPC A61F 5/566; A61B 17/24; A61F 2/02; A61B 2017/248; A61F 2210/009

(57) ABSTRACT

A system for preventing snoring and sleep apnea, the system comprising: a first element (18) and a second element (20) magnetically attracted to each other; wherein the first element (18) is implanted in a patient's tongue (14) that turns to pharynx (16) of the patient; means for continuously adjusting a magnetic attraction between said first element (18) and said second element (20) without relocating the magnetic elements (18, 20); holding means (22), for holding the second element (20), on the exterior front side of the neck of the patient, thereby placing the second element (20) in vicinity with the first element (18); thereby a magnetic attraction of the first and second elements to each other pulls the first element (18) along with the tongue (14) away from the trachea (26), resulting in keeping open the airway of the patient.

### TECHNICAL FIELD

The present invention relates to the field of means for prevention of snoring and sleep apnea.

### BACKGROUND ART

“Sleep apnea is a type of sleep disorder characterized by pauses in breathing or instances of shallow or infrequent breathing during sleep. Each pause in breathing, called an apnea, can last for several seconds to several minutes, and may occur 5 to 30 times or more in an hour.” (From Wikipedia)

These periods of clinical breath cessation are significant if they last for more than 10 seconds each time and occur more than 10 times every hour. Sleep apnea happens during sleep, as it is a lack of muscle tongue in the human upper airway that causes the airway to be blocked. During the day the tongue muscles are sufficient to keep the airway open, allowing for normal breathing.

When an apnea occurs during sleep, the individual usually wakes up, most often with a very loud snore or snort. People that suffer from sleep apnea experience waking episodes many times during the night, and consequently feel very sleepy during the day (“Chronic Fatigue Syndrome”). In addition, it results with high blood pressure.

People that suffer from snoring disturb other people present in a bedroom. Furthermore, such people may avoid sleeping outdoor, and therefore avoid traveling.

FIG. 1 schematically illustrates the upper respiratory tract anatomy of a “healthy person”, i.e., a person that has no airway obstruction.

As illustrated in the drawing, there are two air passages to the lungs: the nasal airway 10, and the oral airway 12. Airways 10 and 12 conjoin at a pharynx 16 on the back side of the tongue 14.

FIG. 2 schematically illustrates the upper respiratory tract anatomy in a person that suffers from airway obstruction.

When the person sleeps, the tongue muscles become loose and as a result the tongue 14 falls down and blocks the air passage at pharynx 16. If the tongue leaves a “thin” air passage, it results in snoring. If the tongue blocks the entire airway, it may result in apnea.

The medicine practice has tried for many years to find a solution to sleep apnea, but so far no substantial advance has been achieved. One way to solve sleep apnea is implanting an electrode in the patient's tongue. The electrode generates an electrical stimulation to the tongue, which results in shrinkage of the tongue's muscles, thereby opening the airway passage at pharynx 16.

Another way to solve the problem is called CPAP, the acronym of Continuous Positive Airway Pressure. It is a treatment that uses mild air pressure to keep the airways open. Presently CPAP is considered as the best treatment for obstructive sleep apnea. A CPAP machine increases air pressure in the throat of a patient so that his breath airway doesn't collapse when sleeping. A CPAP machine uses a mask that is worn by a patient during sleep, which results in substantial inconvenience to a patient.

Another technology uses a device called Mandibular Advancement Device (MAD). It looks much like a mouth guard used in sports. The devices snap over the upper and lower dental arches of a patient. It comprises metal hinges that cause the lower jaw to be eased forward.

Yet another technology is a tongue retaining device. It is used less commonly than MAD. The device is a splint that holds the tongue in place to keep the airway open.

All the methods described above have not yet provided satisfactory solutions to the problem of snort and sleep apnea.

It is an object of the present invention to provide a solution to the above-mentioned and other problems of the prior art.

Other objects and advantages of the invention will become apparent as the description proceeds.

### SUMMARY OF THE INVENTION

In one aspect, the present invention is directed to a system for preventing snoring and sleep apnea, the system comprising: a first element (18) and a second element (20) magnetically attracted to each other; wherein the first element (18) is implanted in a patient's tongue (14) that turns to pharynx (16) of the patient; means for continuously adjusting a magnetic attraction between said first element (18) and said second element (20) without relocating the magnetic elements (18, 20); holding means (22), for holding the second element (20), on the exterior front side of the neck of the patient, thereby placing the second element (20) in vicinity with the first element (18); thereby a magnetic attraction of the first and second elements to each other pulls the first element (18) along with the tongue (14) away from the trachea (26), resulting in keeping open the airway of the patient.

The first element (18) may be magnetic attracted metal (such as iron, nickel, cobalt) while the second element (20) is magnetic, and vice versa. According to one embodiment of the invention, both the first element (18) and the second element (20) are magnetic.

The holding means may be a collar, plaster, bandage, and so on.

According to one embodiment of the invention, the attraction between the elements is adjustable. This can be obtained by adjusting the distance between the attracted elements, their orientation to each other, the intensity of the magnetic field, and so on.

According to one embodiment of the invention, the holding means (22) is a casing comprising a rotatable roller to which the second element (20) is attached, thereby allowing changing an angle of the second element (20) with regard to the first element (18), thereby changing an attraction level of the second element (20) to the first element (18).

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

### BRIEF DESCRIPTION OF DRAWINGS

Preferred embodiments, features, aspects and advantages of the present invention are described herein in conjunction with the following drawings:

FIG. 1 schematically illustrates the upper respiratory tract anatomy of a “healthy person”, i.e., a person that has no airway obstruction.

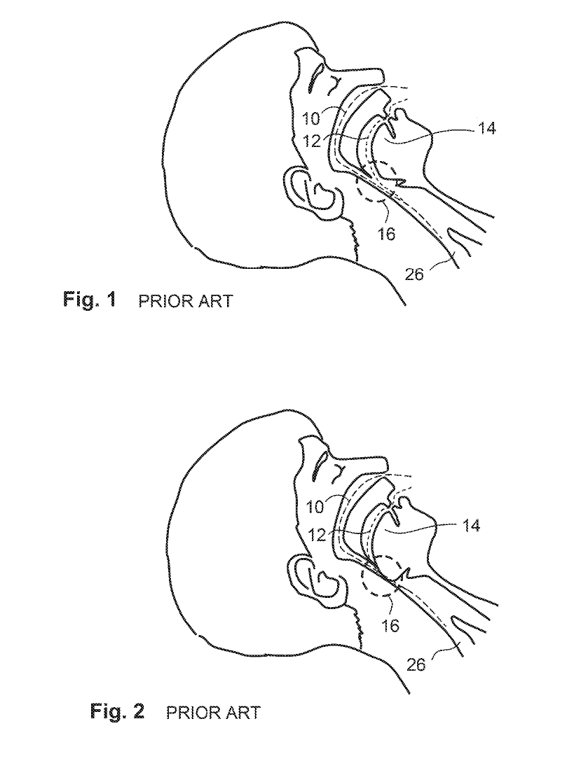

FIG. 2 schematically illustrates the upper respiratory tract anatomy in a person that suffers from airway obstruction.

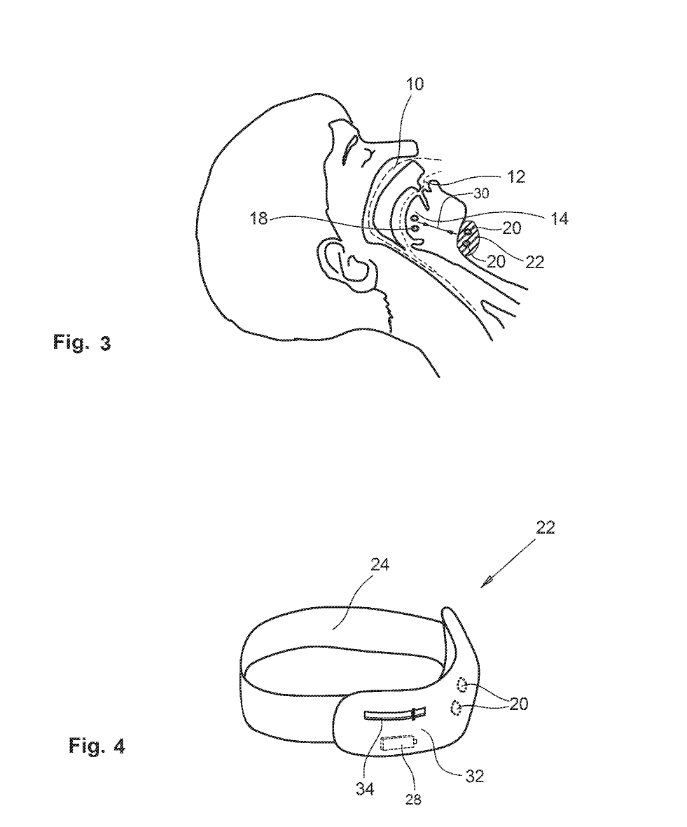

FIG. 3 schematically illustrates a system for preventing snoring and sleep apnea, according to one embodiment of the invention.

FIG. 4 schematically illustrates holding means of a system for preventing snoring and sleep apnea, according to one embodiment of the invention.

Each of FIGS. 5 and 6 schematically illustrates a way of placing straps 24 of the holding means 22 to a patient, according to one embodiment of the invention.

Each of FIGS. 7a and 7b schematically illustrates a state of the holding means 22, according to one embodiment of the invention.

It should be understood that the drawings are not necessarily drawn to scale.

### DESCRIPTION OF EMBODIMENTS

The present invention will be understood from the following detailed description of preferred embodiments (“best mode”), which are meant to be descriptive and not limiting. For the sake of brevity, some well-known features, methods, systems, procedures, components, circuits, and so on, are not described in detail.

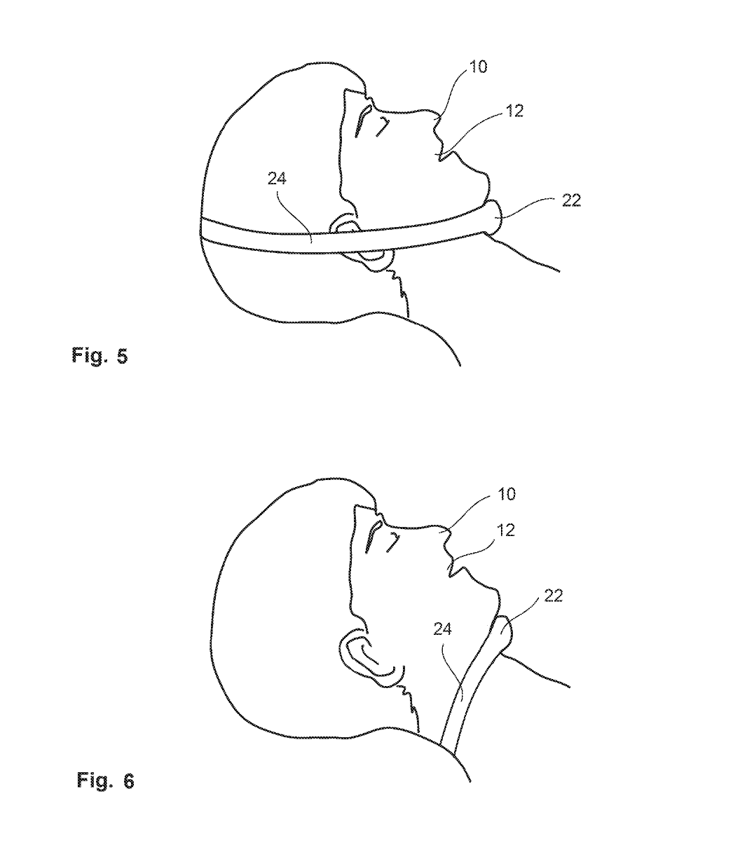

FIG. 3 schematically illustrates a system for preventing snoring and sleep apnea, according to one embodiment of the invention.

The system comprises two elements that are magnetically attracted to each other. The first element, which is marked herein by reference numeral 18, is implanted at the back side of tongue 14 of a patient. Upon use, the second element 20 is disposed at the outer front side of the neck of the patient, close to the first element 18, as illustrated in the figure. Element 20 is held in its place by holding means 22, such as a collar, plaster, bandage, and so on.

Assuming the second element 20 is fixed, i.e., cannot retract, the magnetic attraction pulls element 18, which is disposed at the back side of the tongue, towards the fixed element 20, and as a result, the breath airway opens at pharynx 16 area.

According to one embodiment of the invention, the implanted element 18 is a magnetic attracted metal element, while the fixed element 20 is a magnetic object.

According to another embodiment of the invention, the implanted element 18 is a magnetic element, while the fixed element 20 is a magnetic attracted metal object.

According to yet another embodiment of the invention, both elements 18 and 20 are magnetic, and arranged in such a way that their polarity attracts each other.

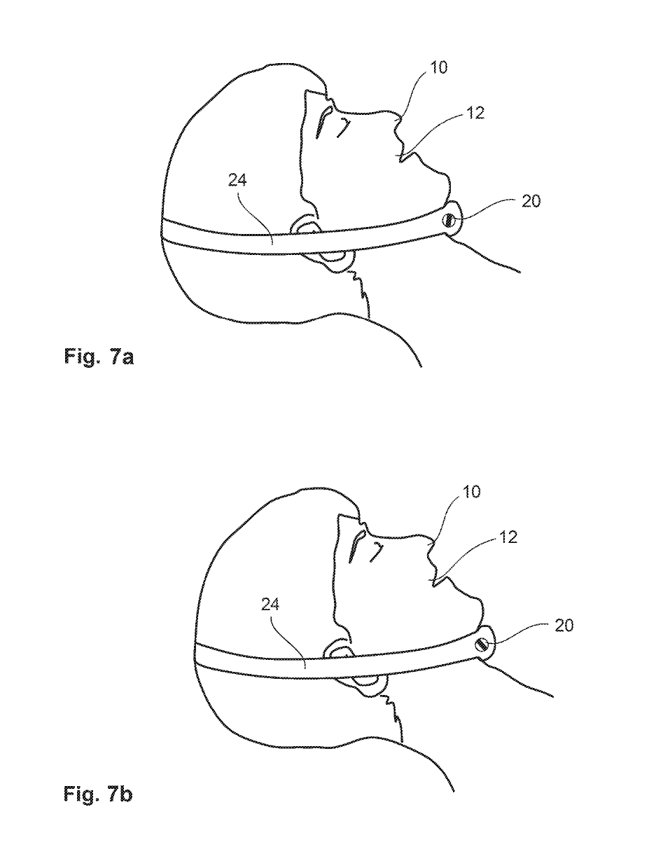

FIG. 4 schematically illustrates an example of holding means 22 of a system for preventing snoring and sleep apnea, according to one embodiment of the invention.

In this example, the holding means 22 is in the form of a collar 24, to which is connected a casing 32, in which a secured element 20 is disposed.

In FIG. 4, the holding means is a collar worn by a patient in the manner illustrated in FIGS. 5 and 6.

Another example of holding means is a bandage or a plaster that attaches element 20 to the external side of the neck of a patient, and may be removed after use.

Adjusting the Intensity of the Magnetic Attraction:

The intensity of the magnetic attraction may be adjusted in several ways. For example, the collar may comprise a plurality of pockets, each disposed at a different distance from the bottom of the collar. This structure allows a user to place element 20 at a different distance from the implanted element 18.

According to one embodiment of the invention, the orientation of element 20 with reference to element 18 is changeable, i.e., the angle of element 20 with reference to element 18. When changing the orientation between elements 18 and 20, the attraction power is changed.

This principle is demonstrated in FIGS. 7a and 7b. Element 20 may be in the form of a roller, in which is disposed a magnet or magnetic attracted metal object, which is attracted to element 18. Upon changing the orientation of object 20, the magnetic attraction between element 20 to 18 is changed.

According to one embodiment of the invention, the magnetic force is generated by electromagnetic means. The magnetic attraction intensity of the electromagnetic means may be adjusted by a slider 34, a knob, and the like, which changes the electric power applied on the electromagnetic element by battery 28.

Of course, these are merely examples, and the magnetic attraction can be adjusted by other means, such as changing the distance between elements 18 and 20, their angles, and so on.

In the figures and/or description herein, the following reference numerals (Reference Signs List) have been mentioned:

numeral 10 denotes a nasal airway of a patient;

numeral 12 denotes an oral airway of the patient;

numeral 14 denotes a tongue of the patient;

numeral 16 denotes a pharynx;

numeral 18 denotes a first element of two elements that are magnetically attracted, wherein element 18 is implanted in the back side of the patient's tongue 14;

numeral 20 denotes a second element of the two elements that are magnetically attracted, wherein element 20 is preferably disposed at the external side of the patient's body, in vicinity to element 18;

numeral 22 denotes a collar, a pouch, and the like, for housing the magnetically attracted element 20;

numeral 24 denotes a strap of the collar;

numeral 26 denotes the trachea of the patient;

numeral 28 denotes a battery, for providing power to element 20, when the magnetic element 20 is an electromagnet;

numeral 30 denotes a magnetic attraction between elements 18 and 20;

reference numeral 32 denotes a casing; and

reference numeral 34 denotes a slider as an example of means for adjusting the intensity of the second magnetic element (20), when the second magnetic element is an electromagnet.

The foregoing description and illustrations of the embodiments of the invention has been presented for the purposes of illustration. It is not intended to be exhaustive or to limit the invention to the above description in any form.

Any term that has been defined above and used in the claims, should to be interpreted according to this definition.

The reference numbers in the claims are not a part of the claims, but rather used for facilitating the reading thereof. These reference numbers should not be interpreted as limiting the claims in any form.

1. A system for preventing snoring and sleep apnea, the system comprising:
a first element (18) and a second element (20), magnetically attracted to each other;
wherein said first element (18) is implanted in a patient's tongue (14) that turns to pharynx (16) of said patient;
means for continuously adjusting a magnetic attraction between said first element (18) and said second element (20) without relocating the magnetic elements (18, 20);
holding means (22), for holding said second element (20), on the exterior front side of a neck of said patient, thereby placing said second element (20) in vicinity with said first element (18);
thereby a magnetic attraction of said first and second elements to each other pulls said first element (18) along with said tongue (14) away from said trachea (26), resulting in keeping open an airway of said patient.

2. A system according to claim 1, wherein said first element (18) is magnetic attracted metal, and said second element (20) is magnetic.

3. A system according to claim 1, wherein said second element (20) is magnetic attracted metal, and said first element (18) is magnetic.

4. A system according to claim 1, wherein said first element (18) and said second element (20) are magnetic.

5. A system according to claim 1, wherein said holding means (22) is a collar.

6. A system according to claim 1, wherein said holding means (22) is a casing comprising a rotatable roller to which said second element (20) is attached, thereby allowing changing an angle of said second element (20) with regard to said first element (18), thereby changing an attraction level of said first second element (20) to said first element (18).

7. A system according to claim 1, wherein said holding means is a bandage.

8. A system according to claim 1, wherein said holding means is a plaster.

9. A system according to claim 1, wherein the magnetic attraction is generated by electromagnetic means.

10. A system according to claim 1, wherein said means for continuously adjusting a magnetic attraction between said first element (18) and said second element (20) comprises a mechanism for changing a orientation of said second element with reference to said first element, wherein said orientation being an angle of said second element (20) with reference to said first element (18).

11. A system according to claim 1, wherein (a) said second element (20) is an electromagnet, and (b) said means for continuously adjusting a magnetic attraction comprises a mechanism for adjusting a magnetic power is a slider, knob, and the like.
